(19) United States
(12) Patent Application Publication
(10) Pub. No.: US 2021/0059856 A1
(43) Pub. Date: Mar. 4, 2021

(54) DEVICES, SYSTEMS AND METHODS FOR THERMAL TREATMENT OF BODY TISSUES

(71) Applicant: Elmedical Ltd., Hod-HaSharon (IL)

(72) Inventors: Uzi ESHEL, Tel-Aviv (IL); Jacob LAZAROVITZ, Hod-HaSharon (IL)

(73) Assignee: Elmedical Ltd., Hod-HaSharon (IL)

(21) Appl. No.: 16/962,258

(22) PCT Filed: Jan. 16, 2019
(86) PCT No.: PCT/IL2019/050060

§ 371 Date: Jul. 15, 2020

Related U.S. Application Data
(60) Provisional application No. 62/617,645, filed on Jan. 16, 2018.

(51) Int. Cl. A61F 7/12; A61M 25/10; A61M 25/00

(52) U.S. Cl. CPC A61F 7/123; A61M 25/1002; A61M 25/003; A61F 2007/0028; A61M 2210/1085; A61M 2210/1042; A61M 2210/1475; A61M 2210/1089

(57) ABSTRACT

Devices, system and methods for heating pelvic floor tissues are provided. The devices of the present invention can be used to apply heat to pelvic floor tissues such as rectal, vaginal, urethral and/or bladder tissues in order to treat disorders associated with such tissues.

### RELATED APPLICATION/S

This application claims the benefit of priority from U.S. Provisional Patent Application No. 62/617,645 filed on Jan. 16, 2018, the contents of which are incorporated herein by reference in their entirety.

### FIELD AND BACKGROUND OF THE INVENTION

The present invention relates to devices, systems and methods for treating body tissues and, more particularly, to systems and methods for uniform thermal treatment of the vagina, rectum, urethra and/or bladder for therapeutic or aesthetic purposes.

There is a wide array of non-bacterial urinary tract inflammatory disorders that are generally less understood, harder to diagnose, and harder to treat successfully. Such disorders include overactive bladder, interstitial cystitis, urinary or fecal incontinence, chronic pelvic syndrome and the like. These disorders are typically accompanied by symptoms such as pelvic pain or discomfort, urinary urge or frequency, bladder pain, and even pain in body regions such as the abdomen, lower back or thighs.

It has been recognized by physicians that applying heat to urinary tract tissues may be helpful in easing the symptoms of such disorders. Heat therapy is typically administered via microwave or fluid-filled balloons.

Thermal treatment has also been applied to body tissues such as the vulvo-vaginal tissue for the purpose of rejuvenation.

Vaginal rejuvenation treatments are typically used for restoring and restructuring vaginal tissue in females with multiple vaginal deliveries stretching the vaginal wall or with excess vaginal and/or labial laxity.

Non-invasive thermal treatment of the vaginal cavity (e.g. Femilift™ or ThermiVa™) typically use a laser or RF energy source to heat and contract collagen fibers present in the submucosal layer of the vaginal wall. Thermal contraction of collagen results in a contraction of collagen-rich spaces and provides a “tightening” effect on overlaying tissue.

While radiation energy approaches such as microwave, RF or laser can be effective in “tightening” or creating new collagen fibers, the energy delivered by such modalities can be difficult to control (in as far as direction and intensity) resulting in non-uniform heating of tissue and the generation of ‘hot spots’ that can damage tissue.

### SUMMARY OF THE INVENTION

According to one aspect of the present invention there is provided a device for intravaginal or intrarectal use comprising a probe sized and configured for intravaginal or intrarectal use, the probe including a handle attached to a proximal end of a cylindrical balloon, the handle having a plurality of conduits for circulating a fluid through said balloon and a support member for supporting the balloon for insertion into a vaginal or rectal canal when the balloon is at least partially inflated via fluid.

According to one aspect of the present invention there is provided a catheter for urinary tract use comprising a shaft having a balloon positioned thereupon, the shaft being configured for placement through a urethra and into a bladder such that when a distal end of the shaft is positioned in the bladder, the balloon is positioned within the urethra, the shaft including at least one inflow conduit for conducting a heated fluid through the balloon and out of an opening at the distal end and into the bladder, and at least one outflow conduit for conducting the fluid from the bladder and out of an opening at a proximal end of the shaft.

The present invention successfully addresses the shortcomings of the presently known configurations by providing a device for uniform thermal treatment of a body cavity such as the vaginal canal, anal canal and rectum and urethra and bladder.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE SEVERAL VIEWS OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 illustrates the vaginal/rectal device connected to an extracorporeal unit including a reservoir, pump and heat exchanger.

FIGS. 2A-8B illustrate various conduit embodiments for a probe region of the device of FIG. 1, shown in expanded (FIGS. 2A-8A) and non-expanded (FIGS. 2B-8B) states.

FIGS. 8C-D illustrate an alternative balloon design for the probe of FIGS. 2A-8B having a Taurus-shaped bulge at the base.

FIG. 9 illustrates the probe of the device of FIG. 1 positioned within a vaginal cavity.

FIGS. 10A-B illustrate the urethra and bladder catheter (referred to herein as dualthermia catheter) positioned in a female urethra and bladder (FIG. 10A) and a male urethra and bladder (FIG. 10B). Bladder—BL, Urethra—UR, Sphincter—SP.

FIGS. 10C-N illustrate cross sectional views of the dualthermia catheter corresponding to regions shown in FIG. 10A.

FIGS. 11A-D illustrate a system including both the vaginal/rectal probe and the dualthermia catheter connected to a single controller (FIG. 11C) and alternative catheter configurations that can be used in place of the dualthermia catheter (FIGS. 11A-B) and alternative vaginal probe (FIG. 11D).

FIG. 12 illustrates the vaginal/rectal probe and the dualthermia catheter positioned in the vagina and urethra-bladder respectively.

### DESCRIPTION OF THE PREFERRED EMBODIMENTS

The present invention is of devices and systems which can be used to thermally treat body cavities. Specifically, the present invention can be used for thermal treatment of the urethra and bladder, a vaginal cavity and rectum alone or in combination in order to treat pelvic floor disorders and/or rejuvenate vaginal tissues.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Approaches for thermally treating pelvic floor disorders are well known in the art and typically employ RF probes, laser or balloon-mediated heating of urinary tract tissues. While these approaches can be somewhat successful they can result in non-uniform heating of tissue which can in turn result in less than optimal treatment and in some cases, tissue damage due to overheating.

While reducing the present invention to practice, the present inventors postulated that thermal treatment of pelvic floor disorders as well as treatment of specific regions (e.g. vagina) can be greatly enhanced if heat is delivered in a uniform manner such that a steady temperature is achieved and maintained throughout these tissues during the course of treatment.

As is further described hereinbelow, the present inventors developed a vaginal/rectal device having a probe that includes an inflatable/fillable chamber (also referred to herein as a “balloon”) that is expandable via a heated fluid circulated therein. The balloon expands to completely occupy the rectum/vaginal cavity thus maximizing thermal contact between the heated fluid and these tissues to provide uniform heating thereof.

The present inventors have also developed a catheter for the uniform and controlled heating of urethral and bladder tissue (referred to herein as “dualthermia catheter”). The vaginal/rectal device and the dualthermia catheter can be used separately or in combination to treat pelvic floor disorders as well as provide cosmetic rejuvenation of vaginal tissue.

Vagina/Rectal Device

According to one aspect of the present invention there is provided a device for thermally treating a body cavity such as an intravaginal cavity or the rectal cavity. As used herein, the term “treatment” encompasses cosmetic treatment such as vaginal rejuvenation, and non-cosmetic (physiological) treatments for reducing stress urinary incontinence, fecal incontinence, sphincter dysfunction and the like.

The present device includes a probe having a handle attached to a proximal end of a cylindrical balloon. In the case of a vaginal device, the balloon is sized and configured for intravaginal use and can be 30-150 mm in length, 10-30 mm in diameter when deflated and 15-80 mm in diameter when fully inflated. In the case of a rectal device, the balloon can be sized and configured for rectal and anal sphincter use and can be 10-150 in length, 10-20 mm in diameter when deflated and 20-60 mm in diameter when fully inflated.

The balloon is preferably fabricated from a compliant material such as silicone, silicone rubber, nylon or the like having a thickness of 100-700 microns and an elastic coefficient of 1-50 MPa.

The balloon can also be fabricated from a pliable non-elastic (non-compliant) or semi-elastic (semi-compliant) material such as nylon or high shore Silicone (Shore A 50 or higher) in which case, the balloon can be fabricated at a final inflation volume and folded against the probe for delivery or it can be primed with a fluid and stretched (non-elastically) to a final volume prior to treatment.

When inflated (filled) with a fluid such as saline or water, the balloon can expand (radially and optionally longitudinally) or unfold to completely fill the vaginal/rectal cavity. Since the balloon can be compliant it will assume the volumetric shape of the vaginal/rectal cavity to maximize contact between the walls of the balloon and the walls of the vaginal/rectal cavity.

Inflation to a pressure of 0.05 to over 1.0 atms can expand the balloon and stretch the balloon wall such that it increases in length by 20-100% and in diameter by 30-300%. The increase in length can be directional, i.e. from the base of the balloon where it is attached to the handle, in a distal direction or it can be bi directional. In the latter case, the balloon walls not attached the base can expand in a proximal direction.

The external surface of the balloon can be smooth or textured to increase contact and thermal conductance.

The handle attached to the balloon includes a plurality of conduits for circulating a fluid through the balloon and a support member for supporting the balloon for insertion through a vaginal/rectal canal when the balloon is at least partially inflated.

Since the balloon is composed of a pliable material it will not have enough longitudinal rigidity for insertion through a vaginal canal even when inflated. The supporting member, which is attached to the handle and extends through a length of the balloon provides the necessary longitudinal rigidity and also carries at least some of the fluid conduits needed for circulating a heated fluid through the balloon. The support member is attached to the base of the balloon and optionally also to the distal end thereof.

In order to expand/fill the balloon with a heated fluid and maintain the heated fluid circulating through the balloon to maintain a uniform temperature, the present device employs one of several conduit arrangements.

Filling a compliant balloon with circulating fluid requires careful planning of the inflow and outflow conduits. If inflow is greater than outflow a balloon can overinflate and conversely, if outflow is greater than inflow the balloon might not fully inflate. This balance is complicated by the tissue walls (e.g. vaginal walls) surrounding the balloon since contact between an inflating balloon and tissue wall can increase the pressure in the balloon and alter flow dynamics.

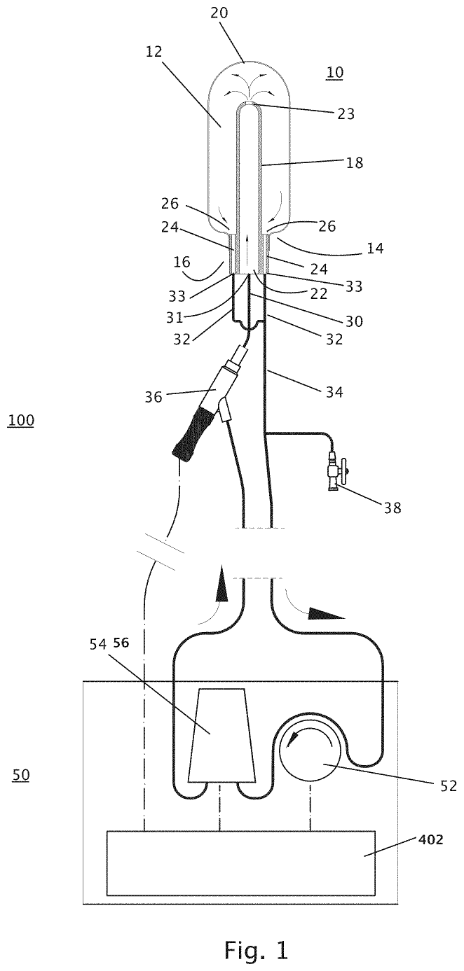

To provide a uniform and steady temperature within the balloon, it should inflate uniformly while maintaining contact with the walls of the vaginal/rectal cavity, the present device includes at least 3 conduits (2 inflow and one outflow or two outflow and one inflow) that circulate fluid through the balloon. Filling of the balloon can be from the balloon distal or proximal end, while emptying is generally effected from an opposite end. The inflow and outflow openings can be in the same plane or they can be rotationally offset from each other (e.g. by 30-90 degrees). By continuously filling the balloon from one end and emptying it from the other (i.e. circulating from distal to proximal), an expansion/filling pressure (of 0.05-1 atms) is maintained within the balloon while fluid is continuously circulated thus maintaining a steady fluid temperature within the balloon and a steady temperature at the vaginal/rectal walls. In addition, the openings of the conduits into, and out of, the balloon can be configured such that pressure changes within the balloon alters opening size thus compensating for over or under-pressure in the balloon. Fluid conduit configurations for providing such functionality are further described hereinbelow with reference to FIGS. 1-8D.

It will be appreciated that in the case of a pliable non-compliant (or semi-compliant) balloon, a substantial back pressure need not be applied since filling of such a balloon (which is more like a sac) would not apply substantial pressure to the fluid traveling therein.

In order to circulate fluid through the balloon, the present device is fluidly connected to an extracorporeal unit that includes a fluid reservoir (e.g. bag), a pump and a heat exchanger (FIG. 1). Controls for the extracorporeal unit (e.g. fluid temperature, fluid flow rate etc.) can be provided on the extracorporeal unit and/or on the handle of the present device.

The fluid within the reservoir is circulated via the pump (e.g. peristaltic pump) through the reservoir and into inflow tubes connected to the inflow conduit(s) of the handle. The fluid is forced through the handle conduits and into the balloon thereby expanding it. Once the balloon reaches an expanded internal pressure, fluid is returned through outflow conduits and outflow tubes connected to the reservoir thereby creating a circulation loop between the balloon and the reservoir.

Referring now to the drawings, FIG. 1 illustrates the present device which is referred to herein as device 10. Device 10 is fluidly connected to an extracorporeal unit 50, device 10 and extracorporeal unit 50 are collectively referred to herein as system 100.

Device 10 includes a balloon 12 attached at a proximal end 14 thereof to a handle 16. Balloon 12 can be fabricated by blow molding or casting from silicone having a Shore A value of 10-50 and a thickness of 300-800 microns. Balloon 12 can be glued or welded to handle 16. Handle 16 is attached to or contiguous with a support member 18 that extends the length of balloon 12. Support member 18 can extend to distal end 20 of balloon 12 to contact an inner wall of balloon 12 (and alternatively be attached, e.g. glued, thereto) or to a region within balloon that is displaced 5-20 mm from distal end 20.

Handle 16 can be cylindrical with dimensions suitable for grasping by a hand of an operator (e.g. 100 mm in length, 30 mm in diameter). Handle 16 can be fabricated from a polymer using well known approaches.

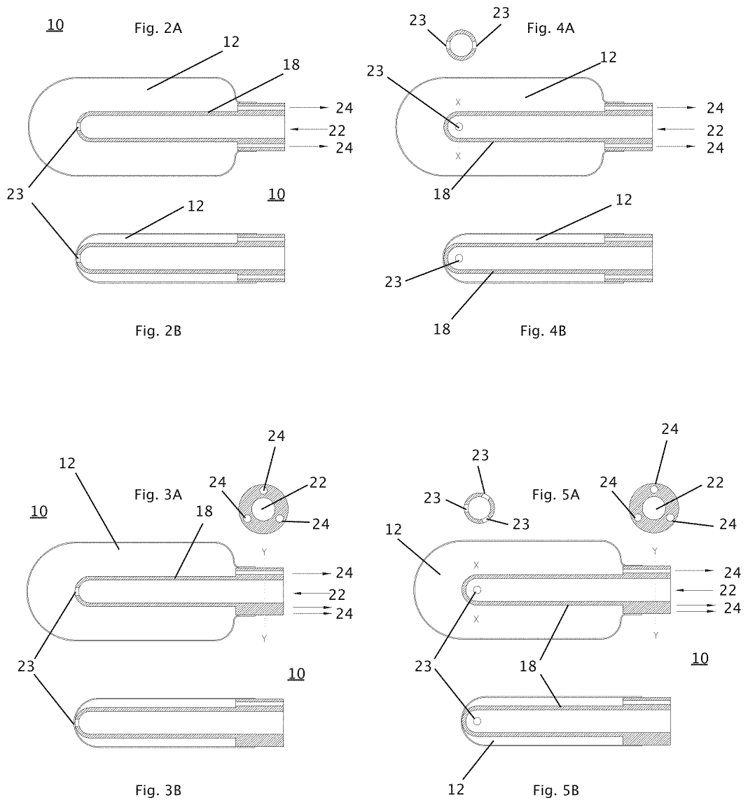

Handle 16 and support member 18 include fluid conduits for circulating a fluid through balloon 12. In the configuration shown in FIG. 1, support member 18 includes a single inflow conduit 22 (having an internal diameter of 2-12 mm) and a single (lateral) opening 23 (with a diameter of 2-5 mm) for providing a fluid to a distal end portion (tip) of balloon 12. Handle 16 includes two outflow conduits 24 each having an opening 26 at or near a base of balloon 12. The internal diameter of each conduit 24 can be 1-4 mm, while the diameter of opening 26 can be 1-4 mm. Alternative configurations of inflow and outflow conduits are described hereinbelow with reference to FIGS. 2A-8B.

Inflow conduit 22 is connected to an inflow tube 30 (through a port 31 in handle 16) while each of outflow conduits 24 is connected—through a port 33 in handle 16—to an outflow tube 32 (which merge to a single outflow tube 34). Inflow conduit 22 can be fabricated from a polymer (e.g. polycarbonate) with an internal diameter of 1-12 mm. Outflow conduit 24 can be fabricated from silicone tubing with an internal diameter of 1-5 mm while outflow conduit 26 an be fabricated from silicone tubing with an internal diameter of 1-5 mm.

Inflow tube 30 and outflow tube 34 are connected to extracorporeal unit 50 which includes a pump 52, a heat exchanger 54 and a controller 402. Inflow tube 30 includes an inline temperature sensor 36 that includes an outer sensor housing and an internal sensor sleeve (condom-like) to separate the temperature sensor from the heated fluid contacting the outer housing. Outflow tube 34 includes a port 38 for priming the system. A syringe is connected to port 38 and air present in device 10 is evacuated using the syringe. Water is then introduced into device 10 via port 38 to inflate balloon 12 and circulate fluid through balloon 12 and heat exchanger 54.

Pump 52 can be a peristaltic pump with a flow rate capacity of 50-500 ml/sec. Heat exchanger 54 can be as described in U.S. Pat. No. 8,940,035.

System 100 is set up and operated as follows. Device 10 is connected to heat exchanger 54 through pump 52 with controller 402 operating both (after priming system 100). Fluid is then circulated through device 10 at a preset rate while monitoring (and modifying if necessary) the fluid temperature. Inflow fluid runs through conduit 22 and into balloon 12 (through opening 23). The fluid then exits balloon 12 through opening 26 and into conduit 24 and then 34 and back to heat exchanger 54.

Device 10 described above includes a single inflow conduit with an opening at or near the distal end of the balloon and two outflow conduits with openings at the proximal end of the balloon. It will be appreciated that this conduit arrangement can be reversed, i.e. two inflow conduits and one outflow conduits and that the openings of such conduits can be positioned anywhere within the balloon. FIGS. 2A-8B described below provide several alternative conduit arrangements of device 10.

FIGS. 2A-8B illustrate several fluid conduit configurations that can be used in device 10 of the present invention.

Circulating a fluid within balloon 12 while maintaining it uniformly expanded around support member such that it contacts the walls of the body cavity/lumen depends on the number of inflow and outflow conduits, their internal diameter and the position and orientation of the conduit openings within balloon 12.

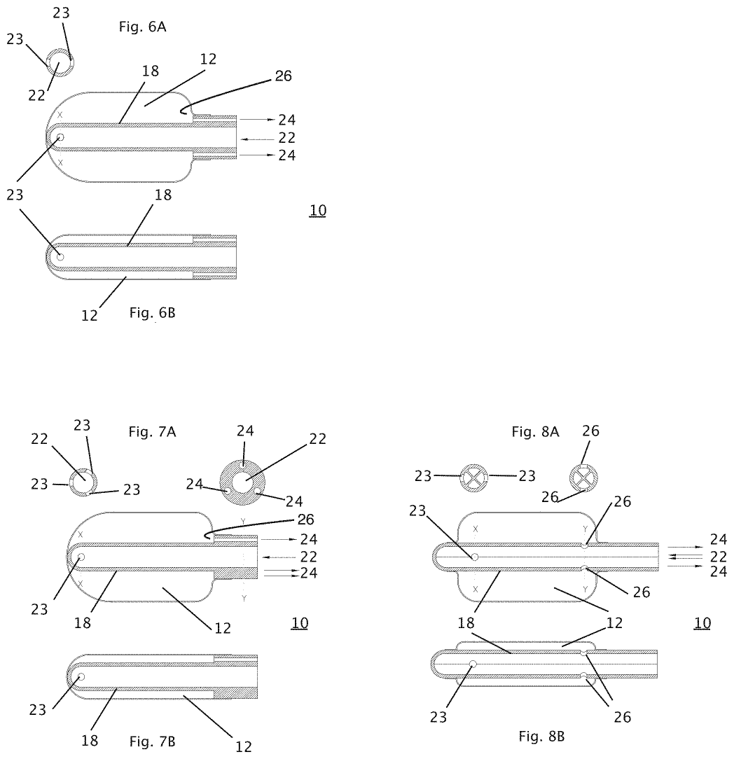

FIGS. 6A-B illustrate an embodiment of device 10 having a single inflow conduit 22 (enclosed by support member 18) and two outflow conduits 24. FIG. 6A illustrates balloon 12 in an expanded state while in FIG. 2B balloon 12 is in a non-expanded state. A cross section of device 10 at support member 18 (plane X-X) showing the arrangement of conduit 22 and the spatial location of openings 23 is provided above FIG. 6A. Inflow conduit has one or more openings 23 at the distal (D) side (also referred to herein as ‘distal end’) of balloon 12 while each of outflow conduits 24 has a single opening 26 at a proximal end of balloon 12. The internal diameter of inflow conduit 22 is 2-6 times larger than the internal diameter of each outflow conduits 24. In this configuration of device 10, support member 18 is attached (glued/welded) to a distal wall of balloon 12.

FIGS. 7A-B illustrate an embodiment of device 10 having a single inflow conduit 22 and three outflow conduits 24. FIG. 7A illustrates balloon 12 in an expanded state while in FIG. 3B balloon 12 is not expanded. A cross section of device 10 showing the arrangement of inflow 22 conduit and openings 26 at support member 18 (plane X-X) and conduits 22 and 24 at handle 16 (plane Y-Y) is provided above FIG. 7A (left and right respectively). Inflow conduit 22 has one or more openings 23 at the distal end of balloon 12 while each of outflow conduits 24 has a single opening 26 at a proximal end of balloon 12. In this configuration of device 10, support member 18 is attached (glued/welded) to a distal wall of balloon 12.

FIGS. 4A-B illustrate an embodiment of device 10 having a single inflow conduit 22 and two outflow conduits 24 (similar to FIGS. 2A-B). In this configuration, opening 23 of inflow conduit 22 is displaced from the distal end of balloon 12 (10-30 mm) when balloon 12 is expanded (FIG. 4A) since support member 18 is not attached to the distal wall of balloon 12. Note that support member 18 (enclosing conduit 22) contacts a distal wall of balloon 12 when deflated (FIG. 4B). A cross section of support member 18 (at plane X-X) is shown above FIG. 4A.

FIGS. 5A-B illustrate an embodiment of device 10 having a single inflow conduit 22 and three outflow conduits 24 (similar to FIGS. 3A-B). In this configuration, opening 23 of inflow conduit 22 is displaced from the distal end of balloon 12 (10-30 mm) when balloon 12 is expanded (FIG. 5A) since support member 18 is not attached to the distal wall of balloon 12. Note that support member 18 (enclosing conduit 22) contact a distal wall of balloon 12 when deflated (FIG. 5B). A cross section of support member 18 (at plane X-X) and at plane Y-Y (handle 16) is shown above FIG. 5A (left and right respectively).

Opening 23 of the configurations shown in FIGS. 4A-8B is directed to a side of balloon 12. In FIGS. 2A-B, which illustrates a device 10 having two outflow conduits 24 and one inflow conduit 22, opening 23 opens towards a distal end of balloon 12. A similar configuration is shown in FIGS. 3A-B which illustrate a device 10 having three outflow conduits 24 and one inflow conduit 22 with opening 23 directed towards a distal end of balloon 12 and an opening 23 directed at a distal end of balloon 12.

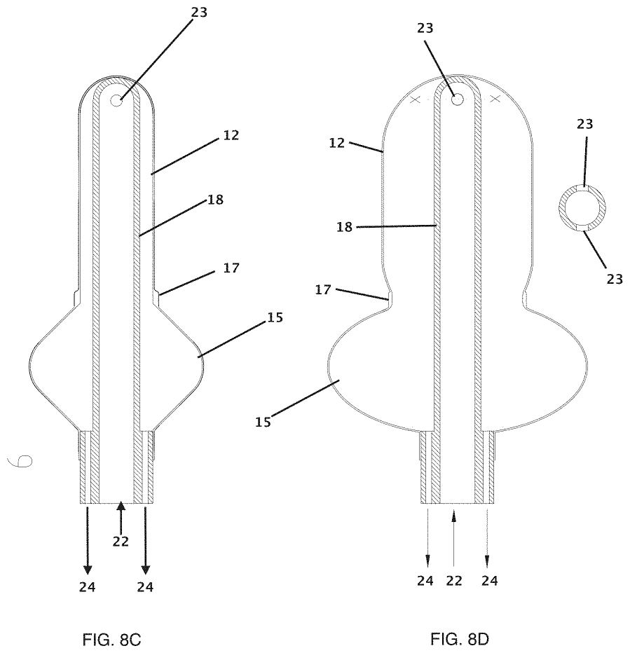

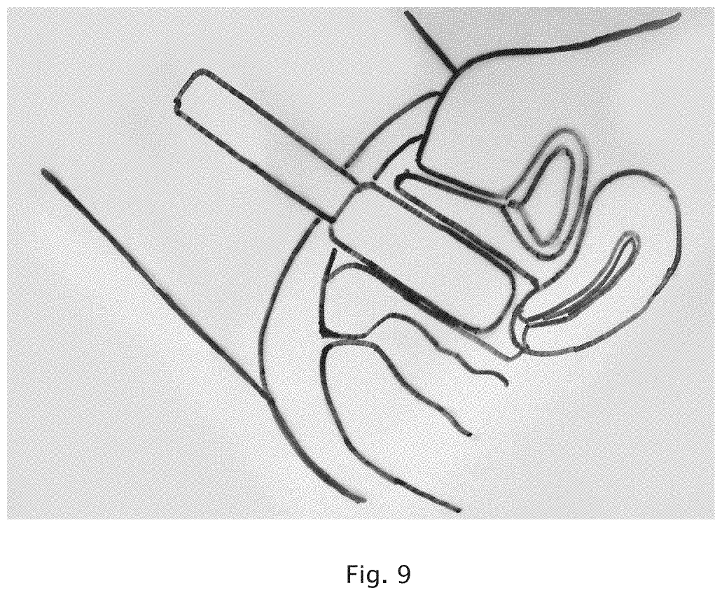

Balloon 12 can further include a toroidal bulge 15 at a base thereof (shown in FIGS. 8C-D and 11D) that can be continuous with balloon 12 or separate therefrom. In the case where bulge 15 is continuous with balloon 12, a thickened band 17 (FIGS. 8C-D) is provided between bulge 15 and the balloon portion distal thereto in order to ensure that both these contiguous sections of balloon 12 maintain the desired shape under inflation. Bulge 15 can be a separate toroidal balloon positioned on handle 16 adjacent to the base of balloon 12. Toroidal bulge 15 can be 30-70 mm in diameter and 10-40 mm in width. Like balloon 12 it can be made from silicone and the like. Bulge 15 can be co-inflated with balloon 12 (sharing the same fluid inflation lumens) or it can include dedicated lumens for inflation/deflation with a heated fluid. When device 10 is positioned in the vaginal cavity, balloon bulge 15 inflated with heated fluid can be used to heat extra-vaginal tissue (vulva tissue such as the labia and the like). Such heating can be effected separately or in conjunction with heating of vaginal cavity via balloon 12. Heating of vulva tissue can be used for aesthetically reducing labia minora and labia majora tissues or as therapy for vulvodynia.

FIGS. 8A-B illustrate an embodiment of device 10 having two inflow conduits 22 enclosed within support member 18 and two outflow conduits 24 enclosed within support member 18. Cross sections shown above FIG. 8A (at support member 18—left and handle 16—right) show the arrangement of inflow 22 and outflow 24 conduits within support member 18.

System 100 of the present invention can be used to thermally treat a vaginal/rectal cavity as follows. Device 10 is first primed by suctioning air out from balloon 12 via port 38. Balloon 12 is then partially filled with 50-200 ml of water which is then circulated through device 10 via pump 52. The partially inflated device 10 is inserted into the vaginal canal. A syringe is used to inflate balloon 12 (through port 38) until it contacts the vaginal/rectal walls and applies pressure thereto without causing discomfort. This procedure can be repeated during treatment. Heated water (at 40-48 degrees Celsius) is then circulated through balloon 12 for 30-60 minutes (with periodic inflation/deflation of balloon 12 via port 38). A portion of balloon 12 extending out of the vaginal/rectal cavity can be manually pumped (squeezed) during the procedure in order to expand/contract the portion of balloon 12 positioned within the vaginal/rectal cavity.

It will be appreciated that although device 10 and system 100 are described above in context with vaginal thermal treatment, device 10 can be configured for treatment of other body cavities such as the rectum. For example, a device 10 having a balloon 12 configured having a diameter and length suitable for use in the anal canal/rectal cavity can be used for uniformly heating rectal tissue and the anal sphincter for the purpose of treating fecal incontinence.

Dualthermia Catheter

As is mentioned hereinabove, the present invention also encompasses a dualthermia catheter capable of simultaneously heating the urethra and bladder of a male or female subject.

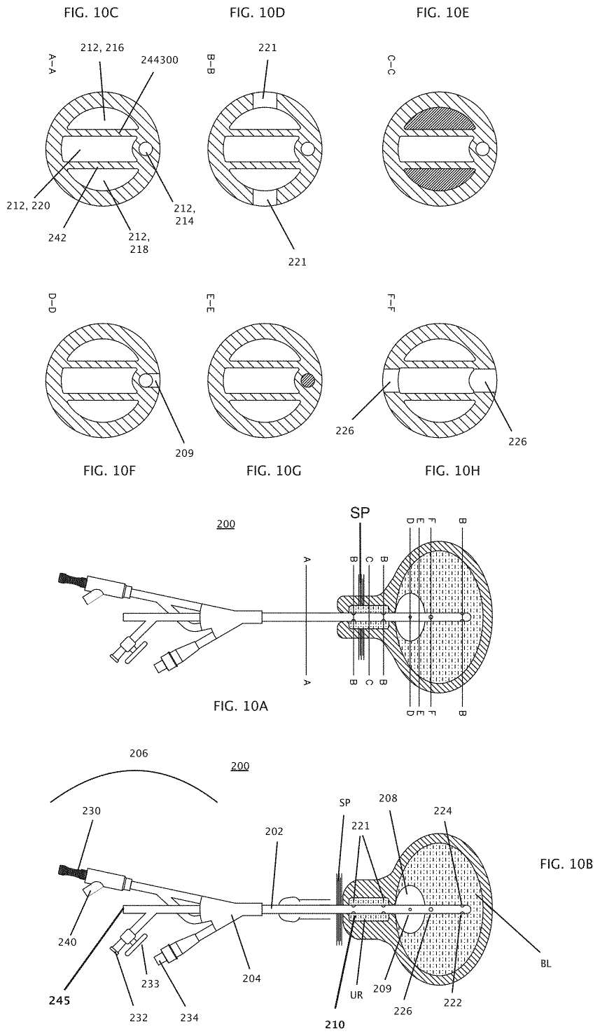

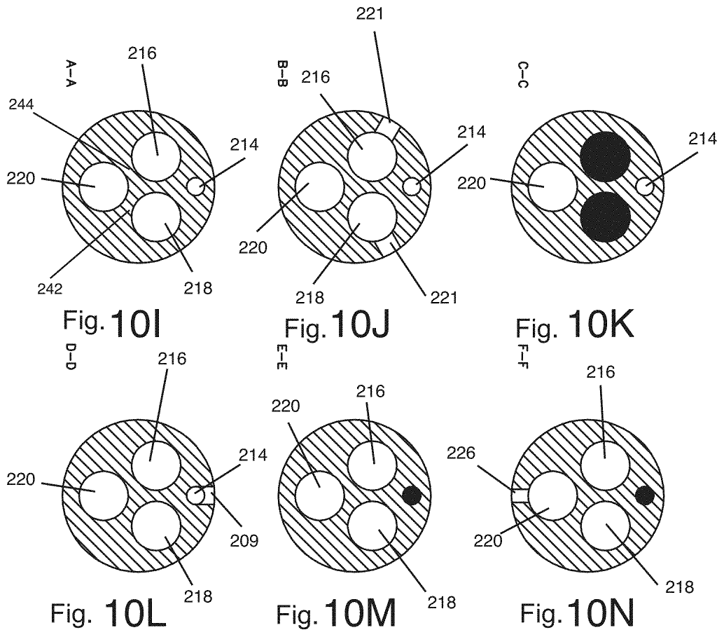

FIGS. 10A-N illustrate the dual-thermia catheter of the present invention which is referred to herein as catheter 200.

FIG. 10A illustrates catheter 200 positioned in a female anatomy, while FIG. 10B illustrates catheter 200 positioned in a male anatomy (Bladder—BL, Urethra—UR, Sphincter—SP, FIG. 10B). Reference numbers and letters utilized herein are shown on FIG. 10B but are also applicable to FIG. 10A.

Catheter 200 includes a shaft 202 connected to a handle 204 supporting a plurality of ports 206. A distal region of shaft 202 includes an anchoring structure 208 (e.g. Foley balloon) for anchoring catheter 200 against the bladder neck to prevent catheter pullout. Shaft 202 also includes a balloon 210 positioned proximally to anchoring structure 208. Balloon 210 is positioned at the urethra when anchoring structure 208 is anchored against the bladder neck region.

Balloon 210 can be fabricated as described above with a length of 20-60 mm and an inflated diameter of 10-20 mm.

Shaft 202 can be cylindrical in shape with a length of 100-350 mm and a diameter of 5-8 mm. Ports 206 are connected to a plurality of conduits 212 running the length of shaft 202. Each of ports 206 is fluidly connected to a specific conduit 212 (conduits reference numbers are shown in FIG. 10C by apply to FIGS. 10D-H).

As is shown in FIGS. 10C-H, shaft 202 includes 4 conduits: a conduit 214 for inflating (via air or liquid) a Foley-type anchoring structure 208 (through opening 209), conduits 216 and 218 for introducing heated fluid into balloon 210 and the bladder and conduit 220 for evacuating the heated fluid from the bladder and balloon 210. Conduits 216, 218 and 220 work together to circulate heated fluid through balloon 210 and the bladder. Conduits 216 and 218 open into balloon 210 at openings 221 (2 shown in FIG. 10D and 4 shown in FIG. 10B) and into the bladder at openings 222 and 224 on distal end of shaft 202. Opening 226 connects the bladder space with conduit 220 to enable drainage of bladder content.

Port 240 is an inflow port for conduits 216 and 218 and port 245 is an outflow port for conduit 220 and includes a valve 233 for priming catheter 200, draining bladder of urine at the beginning of the procedure, increasing/decreasing bladder volumes during the procedure and draining the bladder at the end of the procedure. Port 234 enables inflation of a Foley-type anchoring structure 208 or expansion of a mechanical anchoring structure 208 via, for example, a wire running through conduit 220.

Port 240 includes a temperature sensor probe 230 for monitoring the temperature of the fluid flowing into balloon 210 and the bladder. Sensor 230 can be similar to the temperature sensor described above (with reference to device 10).

Each of conduits 216 and 218 is 1-3 mm in diameter (internal) while conduit 220 can be 1-4 mm in diameter. To enable efficient circulation of fluid through balloon 210 and the bladder and proper uniform inflation of balloon 210 (to maximize contact with the walls of the urethra), the combined fluid flow capacity of conduits 216 and 218 can exceed that of conduit 220 in the case of a compliant balloon and be equal in the case of a pliable non or semi-compliant balloon. Alternatively, within balloon 210, proximal openings 221 can be larger in diameter (or higher in flow) than distal openings 221 so as to create backpressure. For example, proximal openings 221 can be 1.5-2.5 mm (e.g. 2 mm) in diameter, and the distal openings 221 can be 1-2 mm (e.g., 1 mm).

In order to regulate flow and pressure within the catheter and ensure that both balloon 210 and the bladder are completely filled with circulating fluid, walls 242 and 244 isolating conduits 216 and 218 from conduit 220 (respectively), are designed so as to deflect (bow in or out) when a pressure differential exists between conduits 216 and 218 and conduit 220. Walls 242 and 244 can be 0.2-0.6 mm thick and fabricated from the material of catheter shaft 202 or from an elastic/pliable material such as silicone.

Such a pressure differential can exist if the bladder is under or over filled. When under-filled, the flow capacity in conduit 220 may drop thus increasing the flow capacity in conduits 216 and 218 (due to enlargement of lumen). Such increase would result in faster bladder filling. If the bladder overfills, flow and pressure (static) may increase in lumen 220 thereby enabling faster evacuation of the bladder. In any case, this feature of catheter 200 self regulates pressures and flows and enables a more consistent more efficient circulation thorough balloon 210 and the bladder.

An alternative configuration of conduits is shown in FIGS. 10I-N (the positions of cross sections A-A to F-F are shown in FIG. 10A). Shaft 202 includes 4 conduits: a conduit 214 for inflating (via air or liquid) a Foley-type anchoring structure 208 (through opening 209), conduits 216 and 218 for introducing heated fluid into balloon 210 and the bladder and conduit 220 for evacuating the heated fluid from the bladder and balloon 210. Conduits 216, 218 and 220 work together to circulate heated fluid through balloon 210 and the bladder. Conduits 216 and 218 open into balloon 210 at openings 221 (2 shown in FIG. 10J). Opening 226 connects the bladder space with conduit 220 to enable drainage of bladder content (FIG. 10N).

Port 240 (FIG. 10B) is an inflow port for conduits 216 and 218 and port 245 (FIG. 10B) is an outflow port for conduit 220 and includes a valve 233 for priming catheter 200, draining bladder of urine at the beginning of the procedure, increasing/decreasing bladder volumes during the procedure, and draining the bladder at the end of the procedure. Port 234 enables inflation of a Foley-type anchoring structure 208 or expansion of a mechanical anchoring structure 208 via, for example, a wire running through conduit 220.

Port 240 includes a temperature sensor probe 230 for monitoring the temperature of the fluid flowing into balloon 210 and the bladder. Sensor 230 can be similar to the temperature sensor described above (with reference to device 10).

Each of conduits 216 and 218 is 1-3 mm in diameter (internal) while conduit 220 can be 1-4 mm in diameter. To enable efficient circulation of fluid through balloon 210 and the bladder and proper uniform inflation of balloon 210 (to maximize contact with the walls of the urethra), the combined fluid flow capacity of conduits 216 and 218 can exceed that of conduit 220 in the case of a compliant balloon and be equal in the case of a pliable non or semi-compliant balloon. Alternatively, within balloon 210, proximal openings 221 can be larger in diameter (or higher in flow) than distal openings 221 so as to create backpressure. For example, proximal openings 221 can be 1.5-2.5 mm (e.g. 2 mm) in diameter, and the distal openings 221 can be 1-2 mm (e.g., 1 mm).

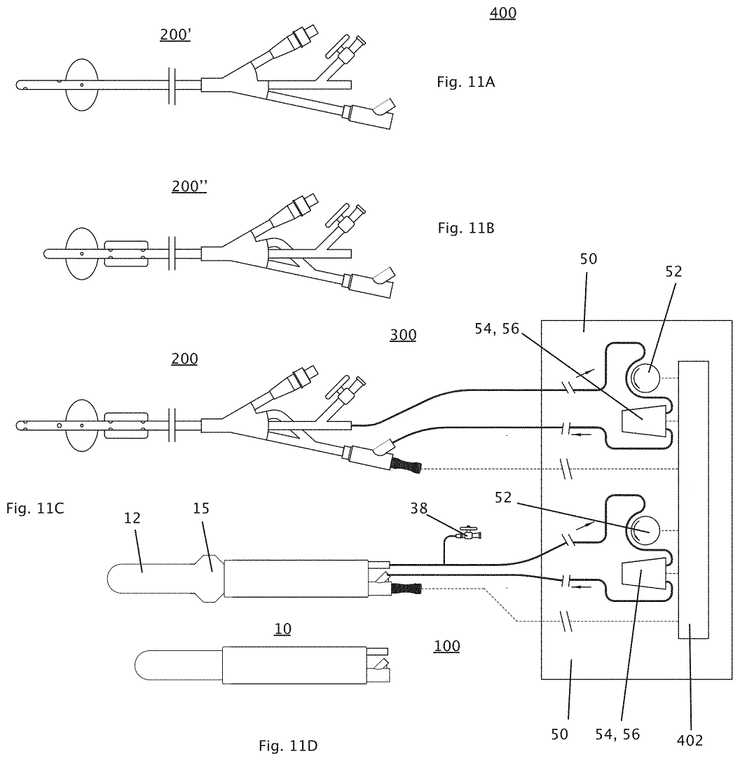

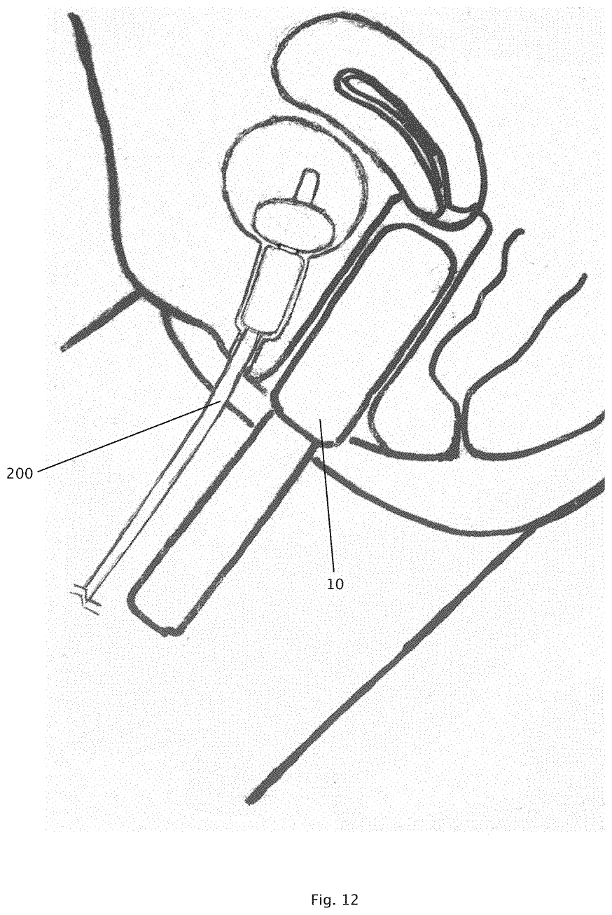

Catheter 200 can be connected to an extracorporeal unit 50 which includes a pump 52, a heat exchanger 54 and a reservoir 56 to form system 300 (shown in FIG. 11). Port 240 can be connected to the fluid outflow lines of reservoir 56. Port 245 can be connected to the inflow line into pump 52.

Pump 52 can be a peristaltic pump as described hereinabove with reference to system 100.

Catheter 200 can be used to thermally treat urethral and bladder tissues as follows.

Catheter 200 is primed as described above for device 10 with the exception that the opening into the bladder is blocked off (via for example a cover or overtube) to prevent outflow from this opening. Primed catheter 200 is then inserted through the urethra and into the bladder and balloon 208 is inflated and catheter 200 is pulled back (in proximal direction) until balloon 208 anchors against the bladder neck with balloon 210 positioned within the urethra. Fluid is then circulated through balloon 210 and bladder while maintaining balloon 210 inflated and the fluid at a steady preset temperature (40-48 degrees Celsius). The volume of fluid circulated through balloon 210 and the bladder is adjusted to the patient (set at a volume below a sensation of pain, e.g., less than a volume of the bladder that creates a painful sensation in the subject).

System for Uniform Heating of the Pelvic Floor

The above described rectal/vaginal device and dualthermia catheter can be used to simultaneously heat the urethra and/or bladder and the rectum/vagina in order to more uniformly heat the pelvic floor. Such uniform heating can provide benefits to treatment of urethra/bladder-related disorders as well as rectal/vaginal -related disorders.

FIGS. 11A-D and 12 illustrate system 400 which includes systems 100 and 300 operated under a single controller 402.

FIGS. 11A-B illustrate alternative configurations of catheter 200 (designated as catheters 200′ and 200″) that can be used in place of catheter 200 in system 400.

FIG. 11A illustrates catheter 200′ which does not include the urethral balloon and as such it is only functional in circulating a heated fluid through a urinary bladder. FIG. 11B illustrates catheter 200″ which includes the urethral balloon but no openings for fluid to be circulated through the urinary bladder.

Catheters 200′ and 200″ can be used in place of catheter 200 in indications that do not require additional heating of cavity-adjacent structures.

By simultaneously heating both urethra and bladder and the vaginal/rectal cavity and surrounding tissues, system 400 of the present invention can uniformly heat the entire pelvic floor and provide effective treatment to disorders associated therewith. Such combined heating would be particularly useful for treating pelvic floor disorders such as overactive bladder, interstitial cystitis, chronic pelvic pain syndrome and the like.

As used herein the term “about” refers to ±10%.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A device for intravaginal or intrarectal use comprising a probe sized and configured for intravaginal or intrarectal use, said probe including a handle attached to a proximal end of a cylindrical balloon, said handle having a plurality of conduits for circulating a fluid through said balloon and a support member for supporting said balloon for insertion into a vaginal or rectal canal when said balloon is at least partially inflated via said fluid.

2. The device of claim 1, wherein said support member extends a full length of said balloon when deflated.

3. The device of claim 2, wherein a first conduit of said plurality of conduits extends a length of said support member.

4. The device of claim 3, wherein said first conduit opens at a distal end of said balloon.

5. The device of claim 4, wherein a second conduit of said plurality of conduits opens at a base of said balloon.

6. The device of claim 5, wherein a flow through said first conduit is equal to said flow through said second conduit.

7. The device of claim 1, wherein said plurality of conduits include at least one conduit for filling said balloon and at least two conduits for emptying said balloon.

8. The device of claim 1, wherein said plurality of conduits include at least one conduit for emptying said balloon and at least two conduits for filling said balloon.

9. The device of claim 1, wherein said balloon is a compliant balloon.

10. The device of claim 9, wherein said compliant balloon is a silicone balloon.

11. The device of claim 10, wherein said silicone balloon is 50-150 mm in length and 15-80 mm in diameter when inflated to a pressure of up to 1 atm.

12. The device of claim 1, wherein said handle includes ports for connecting said plurality of conduits to an external fluid source.

13. The device of claim 2, wherein a distal end of said balloon is attached to said support member.

14. The device of claim 1, wherein said balloon is configured for elastically stretching both longitudinally and radially.

15. The device of claim 1, wherein a proximal portion of said balloon includes a toroidal bulge.

16-17. (canceled)

18. A catheter for urinary tract use comprising a shaft having a balloon positioned thereupon, said shaft being configured for placement through a urethra and into a bladder such that when a distal end of said shaft is positioned in said bladder said balloon is positioned within said urethra, said shaft including at least one inflow conduit for conducting a heated fluid through said balloon and out of an opening at said distal end and into said bladder, and at least one outflow conduit for conducting said fluid from said bladder and out of an opening at a proximal end of said shaft.

19. The catheter of claim 18, wherein a fluid flow capacity of said inflow conduit is at least 25% higher than that of said outflow conduit.

20. The catheter of claim 18, comprising two inflow conduit arranged coaxially around said outflow conduit.

21. The catheter of claim 18, comprising two inflow conduit and one outflow conduit arranged in a circle.

22. The catheter of claim 18, wherein a wall separating said inflow conduit from said outflow conduit is elastic such that a pressure differential between said inflow conduit and said outflow conduit deflects said wall.

23-25. (canceled)
